(19) United States
(12) Patent Application Publication
(10) Pub. No.: US 2024/0177628 A1
(43) Pub. Date: May 30, 2024

(54) System and Method for Predicting Degree of Obesity Based on Growth and Development Data of Infants Using Growth Prediction AI Model

(71) Applicant: Kaii Company Inc., Daejeon (KR)

(72) Inventors: Byoung-Hee Han, Gwangmyeong-si (KR); Miri Jeong, Seoul (KR); Ho-Chung Chung, Daejeon (KR)

(21) Appl. No.: 18/070,975

(22) Filed: Nov. 29, 2022

(51) Int. Cl. G09B 23/28; G16H 20/60

(52) U.S. Cl. CPC G09B 23/28; G16H 20/60

(57) ABSTRACT

In a method of predicting a degree of obesity based on growth and development data of infants or children using a growth prediction AI model according to one embodiment of the present disclosure, the method includes: acquiring first data that is body information data including gender, height, and weight of infants or children, and storing the first data in a database; acquiring second data that is physical activity data for infants or children, and storing the second data in the database; normalizing and categorizing the first and second data; predicting a degree of obesity after n months through a growth curve of the growth prediction AI model based on the normalized first and second data; reacquiring the first and second data after the n months and storing the reacquired first and second date in a database; training the growth prediction AI model based on the predicted degree of obesity and the reacquired first data; predicting a degree of obesity after m months through the trained growth prediction AI model; and analyzing a risk of obesity based on the predicted degree of obesity.

### BACKGROUND OF INVENTION

### Field of the Invention

The present disclosure relates to a system and method for predicting a degree of obesity based on growth and development data of infants and children (hereinafter referred to as “infants”), and specifically, relates to a system and method for predicting more accurate and standardized obesity risk through a growth prediction artificial intelligence (AI) model using physical measurement data and physical activity data, rather than a medical diagnostic method.

### Related Art

Obesity in infants refers to a relatively obese state compared to the same peer group, and a body mass index (BMI) determined by height and weight is generally known as a general and easy way to determine the degree of obesity.

Obesity is divided into primary obesity caused by genetic or environmental factors and secondary obesity caused by central nervous system abnormalities, endocrine diseases, and drugs, and more than 99% of obesity is primary obesity. In fact, when both parents are obese, their children are 70 to 80 percent likely to be obese, and children's risk of obesity is known to double when their mothers are obese. Recently, due to westernization of dietary lifestyles and excessive education, the prevalence of obesity is increasing as the activity of adolescents decreases rapidly.

In particular, obesity in children and adolescents, including infants, seems to have increased sharply due to a decrease in physical activity due to the COVID-19 incident in 2020. According to the National Health Insurance Service, compared to the first half of 2019, before COVID-19, the amount of obesity treatment for infants under the age of 9 in the first half of 2021 increased by 45.3%, and the amount of obesity treatment for teenagers also increased by 29.6%.

Obesity in children and adolescents, including infants, leads to obesity in adulthood and becomes a factor in the early development of metabolic diseases such as cardiovascular disease and diabetes. Furthermore, Obesity affects the psychosocial development of children and adolescents, so that problems due to obesity such as decreased self-esteem, depression, alienation from peers, and emotional anxiety have also been reported.

Despite such situations, the seriousness of the problem of obesity in infants has not yet been properly recognized. Moreover, until now, only through traditional medical services, where doctors, who are clinical experts, analyze growth and development data such as height and weight through infants' direct visits to hospitals and consult with the infants and their guardians, people can get advice on the risk of obesity in the future for infants. According to the traditional service methods, since experts need to diagnose and explain obesity themselves, there is a limit to monitoring a large number of subjects, and a gap occurs depending on each expert's obesity screening clinical ability.

Accordingly, there is a need for a system capable of preventing diseases such as obesity in adulthood and high blood pressure by developing a standardized and systematized infant obesity model to identify infants who are currently obese or at high risk of obesity at an early stage and take appropriate measures.

### SUMMARY

In view of the above, the present disclosure provides a system and method capable of identifying infants with a high risk of obesity early by providing a method of predicting a degree of obesity or obesity risk in infants at a predetermined time point using physical information data such as a height and weight of infants and physical activity data, which is data on endurance, agility, balance, and quickness.

The present disclosure also provides a system and method capable of more accurately predicting a degree of obesity by adjusting weights of prediction variables from a predicted value of an infant's obesity at a predetermined time point and an actual obesity derived from the infant's physical information data obtained at the predetermined time point.

Other objects, particular advantages, and novel features of the present disclosure will become more apparent from the following detailed description and preferred embodiments taken in conjunction with the accompanying drawings.

In a method of predicting a degree of obesity based on growth and development data of infants using a growth prediction artificial intelligence (AI) model according to one embodiment of the present disclosure, the method comprises: acquiring first data that is body information data including gender, height, and weight of infants or children, and storing the first data in a database; acquiring second data that is physical activity data for infants or children, and storing the second data in the database; normalizing and categorizing the first and second data; predicting a degree of obesity after n months through an infant growth curve of the growth prediction AI model based on the normalized first and second data; reacquiring the first and second data after the n months and storing the reacquired first and second date in a database; training the growth prediction AI model based on the predicted degree of obesity and the reacquired first data; predicting a degree of obesity after m months through the trained growth prediction AI model; and analyzing a risk of obesity based on the predicted degree of obesity.

According to another embodiment of the present disclosure, the predicted degree of obesity is a value obtained by calculating a predicted value based on the first data and a predicted value based on the second data, and adding the predicted values according to respective weights, and the training of the growth prediction AI model includes comparing the predicted degree of obesity with the reacquired first data, and adjusting the weights used for predicting the degree of obesity according to the comparison result. The weights are ratios of the respective predicted values based on the first data and the second data to the predicted degree of obesity.

According to still another embodiment of the present disclosure, the second data includes data on endurance, agility, balance, and quickness.

According to still another embodiment of the present disclosure, the categorized information and/or the analyzed obesity risk information is provided in a visualized form on a display to a user. The method further comprises recommending a diet for infants or children based on the categorized information or the analyzed obesity risk information.

According to still another embodiment of the present disclosure, the first and second data are acquired periodically, and the period includes annually, quarterly, monthly, weekly, and the like.

A system for predicting a degree of obesity based on growth and development data of infants using an artificial intelligence algorithm according to still another embodiment of the present disclosure, comprises: a terminal for providing body information data including gender, height, and weight of a user and physical activity data of the user; and an obesity degree prediction server, connected to the terminal through a network, for analyzing the body information data and the physical activity data provided from the terminal to predict degree of obesity at a predetermined time point, and providing information on the degree of obesity to the terminal, wherein the obesity degree prediction server includes: a storage that stores the data provided from the terminal in a database; an obesity degree prediction operator that learns the data in the storage through artificial intelligence (AI) to predict the degree of obesity of the user at the predetermined time point; and an obesity degree information providing unit that provides the information on the degree of obesity predicted by the obesity degree prediction operator to the terminal.

According to still another embodiment of the present disclosure, the obesity degree prediction operator includes: a first prediction unit that predicts body information at a predetermined time point based on the body information data from the terminal; a second prediction unit that predicts body information at the predetermined time point based on the body activity data from the terminal; and a weight determination unit that determines a weight assigned to each of the predicted values of the first prediction unit and the second prediction unit, and wherein the degree of obesity at the predetermined time point can be predicted based on the predicted values from the first prediction unit and the second prediction unit and the weight from the weight determination unit.

According to still another embodiment of the present disclosure, a computer-readable recording medium storing a program operating to perform the above-described method according to the present disclosure and a program stored in the recording medium are provided.

According to the embodiments of the present disclosure as described above, it is possible to identify infants with a high risk of obesity early by providing the method of predicting a degree of obesity or obesity risk in infants at a predetermined time point using physical information data such as height and weight of infants and physical activity data, which is data on endurance, agility, balance, and quickness.

Further, it is possible to more accurately predict degree of obesity by adjusting weights of prediction variables from the predicted value of an infant's obesity at a predetermined time point and the actual obesity derived from the infant's physical information data obtained at the predetermined time point.

In addition, the present disclosure can be applied as an AI screening model for large-scale infant health care projects such as infant physical examination and health checkup, and can be used as an obesity risk clinical decision system and a patient management system for hospitals such as children's hospitals, and growth and development clinics.

### BRIEF DESCRIPTION OF THE DRAWINGS

FIG. 1 is a flowchart illustrating an infant's obesity degree prediction process according to one embodiment of the present disclosure.

FIG. 2 is an exemplary diagram of an information source capable of obtaining body data according to one embodiment of the present disclosure.

FIG. 3 is a distribution diagram of body mass indexes according to one embodiment of the present disclosure.

FIG. 4 is a distribution diagram in which the average of the body mass indexes of FIG. 3 is standardized to 100.

FIG. 5 is a distribution diagram showing a normalized flexibility item.

FIG. 6 is a distribution diagram showing a normalized endurance item.

FIG. 7 is a distribution diagram showing a normalized agility item.

FIG. 8 is a distribution diagram showing a normalized balance item.

FIG. 9 is a distribution diagram showing a normalized quickness item.

FIG. 10 is a distribution diagram showing normalization by converting the sum of the normalized values of FIGS. 5 to 9 into 0 to 100 points.

FIG. 11 is a table showing a correlation between physical information data and physical activity data.

FIG. 12 is a diagram illustrating an obesity degree prediction process according to one embodiment of the present disclosure.

FIG. 13 is a diagram illustrating an example of providing analysis data of information on the height of an infant to a user according to one embodiment of the present disclosure.

### DESCRIPTION OF EXEMPLARY EMBODIMENTS

Terms and words used in the present disclosure described below should not be construed as conventional or dictionary meanings, and should be construed as meanings and concepts conforming to the technical idea of the present disclosure based on the principle that the inventor can appropriately define his or her disclosure as a concept to describe it in the best way.

Accordingly, the configurations of the embodiments described in the present specification and shown in the drawings are only preferred embodiments of the present disclosure and do not represent all technical ideas of the present disclosure, so it should be understood that there may be various equivalents and modifications that can replace them at the time of the present application.

Hereinafter, a method and system for predicting an infant's obesity degree according to one embodiment of the present disclosure will be described in detail with reference to the accompanying drawings.

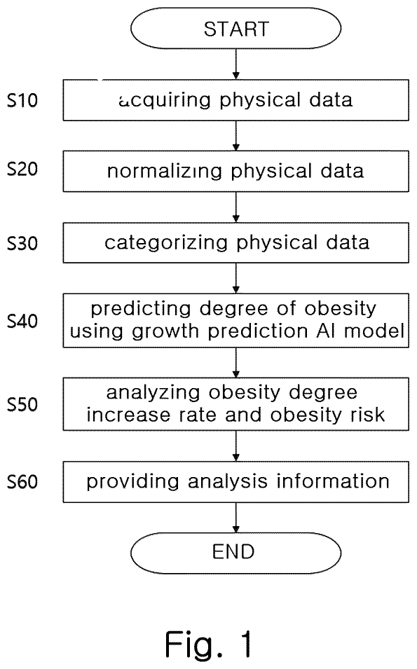

FIG. 1 is a flowchart illustrating an infant's obesity degree prediction process according to one embodiment of the present disclosure.

First, an infant's physical data is acquired and stored in a database (S10). The physical data includes physical information data including gender, height, and weight, and physical activity data such as flexibility, endurance, agility, balance, and quickness. The physical information data may further include any physical data, such as body mass index (BMI), body fat, muscle mass, and thigh circumference, which may be measured using an in-body device, as well as gender, age (monthly age). In the present embodiment, the physical activity data includes data on flexibility, endurance, agility, balance, and quickness, but is not limited thereto.

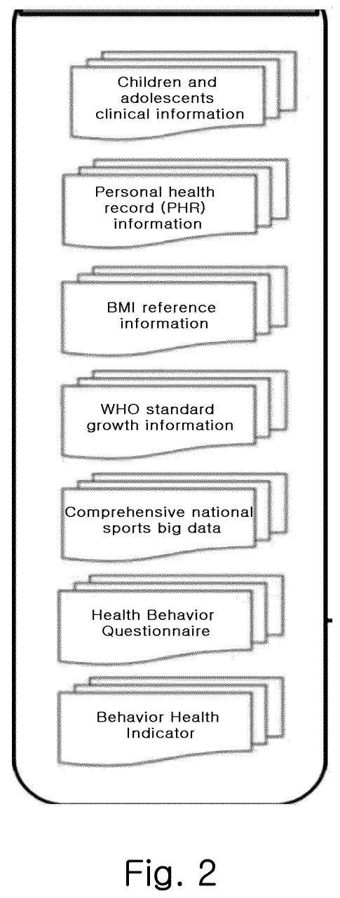

The physical information data and the physical activity data of infants need to be stored in a database in advance for statistical analysis or generation and training of a growth prediction AI model to be described below. When the physical data cannot be directly obtained from infants, information as those shown in FIG. 2 may be used.

The physical information data and the physical activity data are normalized (S20).

An example of normalization using data on infant checkups in Seoul for three years from 2017 to 2019 will be described. The number of samples was 19,513, and the analysis targets were age (monthly age), height, weight, flexibility, endurance, agility balance, and quickness, and when any of the above analysis targets did not have data, it was excluded from the analysis. Flexibility was evaluated by the degree of waist bending, endurance was evaluated by the time to maintain a V-shaped posture, agility was evaluated by a standing long jump, balance was evaluated by the time standing on one foot, and quickness was evaluated by the round-trip time in a predetermined section. However, the present disclosure is not limited to the above, and there may be various evaluation methods.

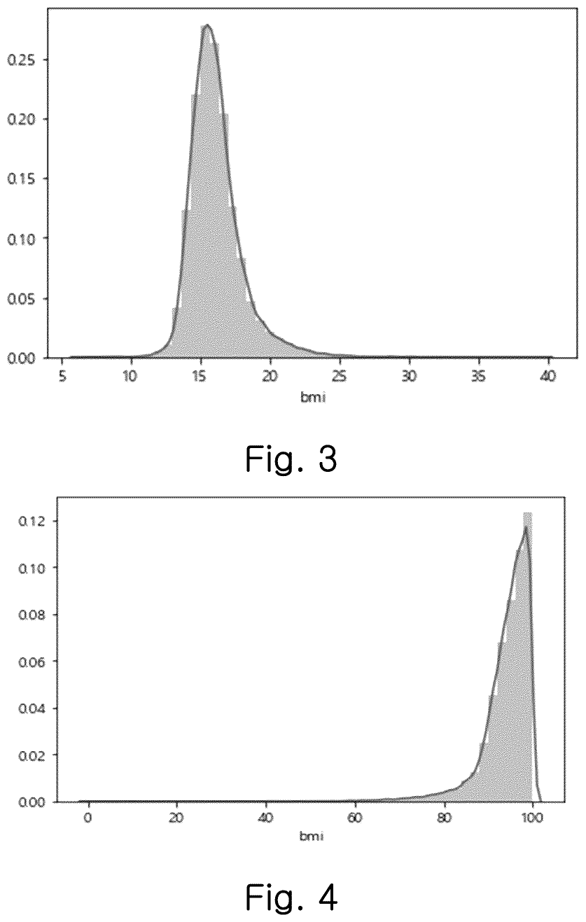

FIG. 3 is a distribution diagram of body mass indexes (BMIs), and FIG. 4 is a distribution diagram in which an average of the body mass indexes is standardized to 100. The average age is 65 months, the average height is 111 cm, the average weight is 19.5 kg, and the average BMI is 15.8. Since the actual body mass index differs depending on the monthly age and gender, the actual body mass index needs to be considered, but it is not considered in the present embodiment.

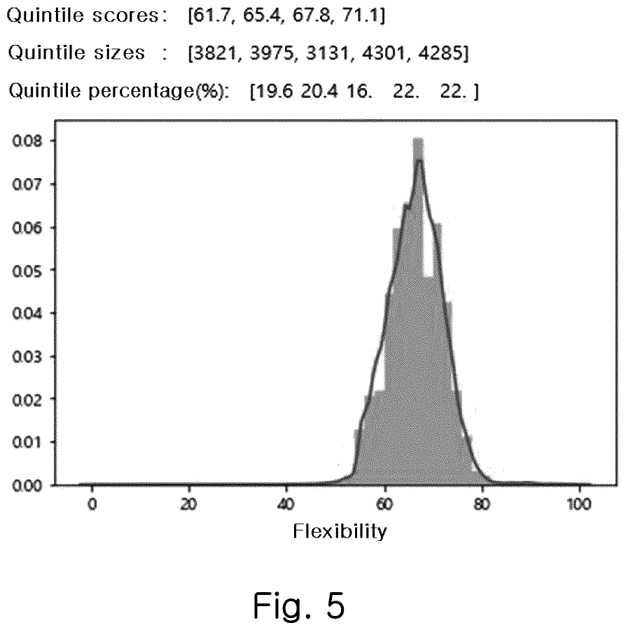

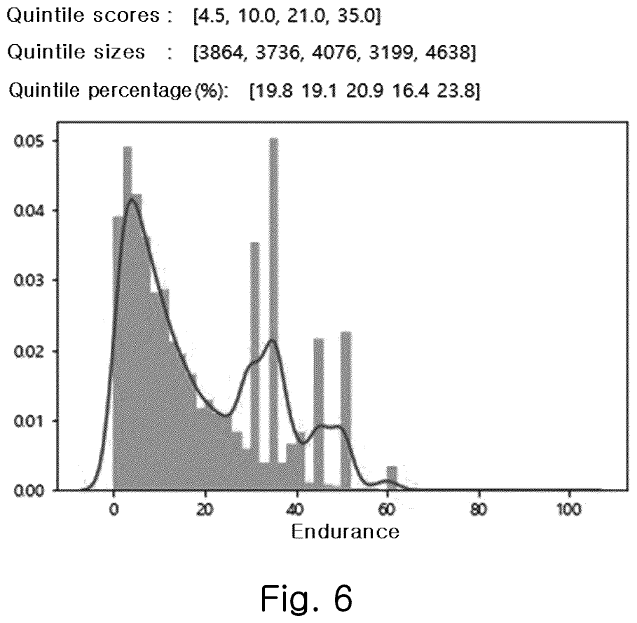

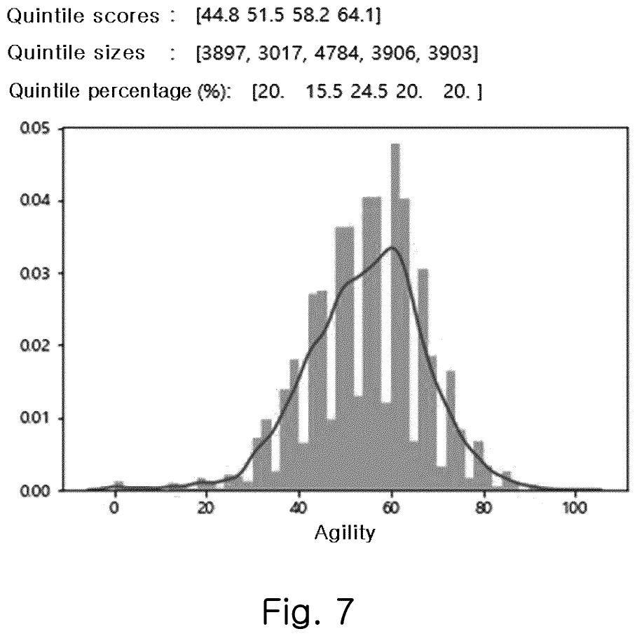

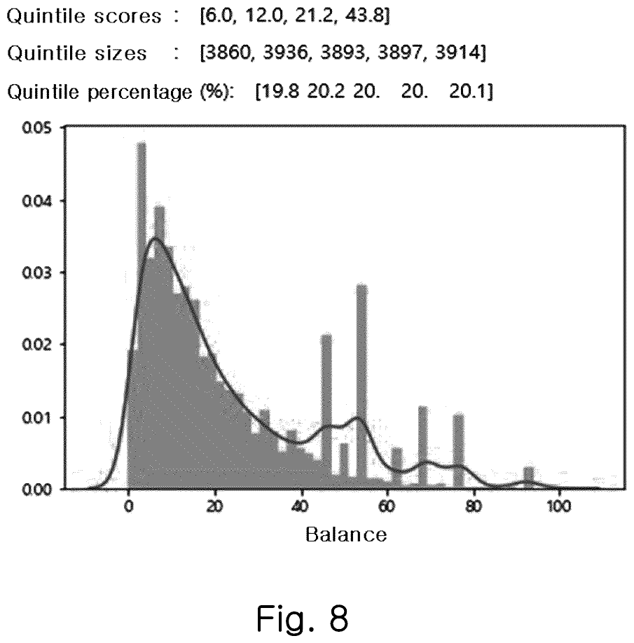

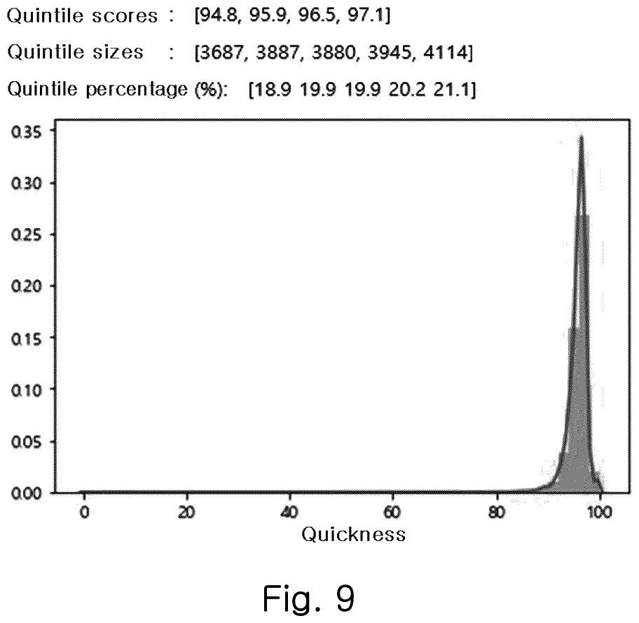

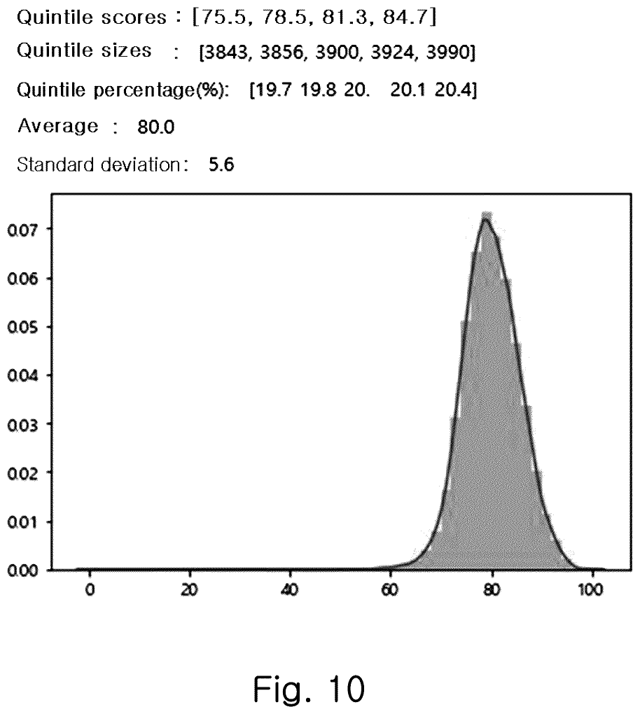

FIGS. 5 to 9 are distribution diagrams showing five items of flexibility, endurance, agility, balance, and quickness, which are physical activity data, normalized to (0,1). In the case of agility, the inverse was made so that the high score was excellent. The quintiles (20% divided by 5 sections) are also displayed. FIG. 10 is a distribution diagram that converts the sum of the normalized values of flexibility, endurance, agility, balance, and quickness into 0 to 100 points and normalizes them to (0,1).

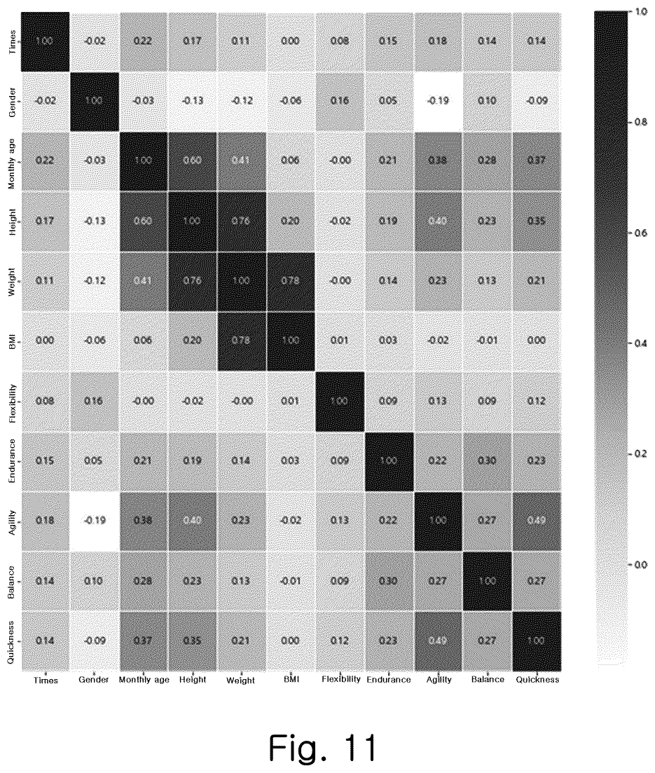

FIG. 11 is a table showing a correlation between physical information data and physical activity data. As shown in the table in FIG. 11, the body information data (monthly age, height, and weight) show a relatively high correlation of 0.41 to 0.76. That is, as the monthly age increases, the height and weight increase, and in particular, the height shows a higher correlation. Among the physical activity data, endurance, agility, balance, and quickness, excluding flexibility, show some correlation with physical information data, that is, height or weight. Based on such correlations, it can be understood that the final prediction value for degree of obesity is determined by determining weights of values of degree of obesity predicted based on the physical information data and the physical activity data, respectively.

In the present embodiment, the normalized value of each data may be loaded into an AI model that classifies data into 5 stages (very good, good, average, attention, need for improvement) to execute a classification procedure (S30), and then may be provided in a form where the average result value for the age group, average ratings and individual results are visualized. In addition, appropriate physical activity or diet may be provided as recommended information depending on the classification.

The method of analyzing the body information data and the physical activity data may be performed by an optimal category classification method and a statistical analysis method by AI.

With the statistical method, the position compared to the average of each age for physical development status is calculated, the sum of five normalized data on physical activity ability is converted into 0 to 100 points, and the quintiles (divided into 5 sections of 20%) are analyzed.

Next, with the normalized data, a degree of obesity at a predetermined time point is predicted using the growth prediction AI model through the infant growth curve (S40).

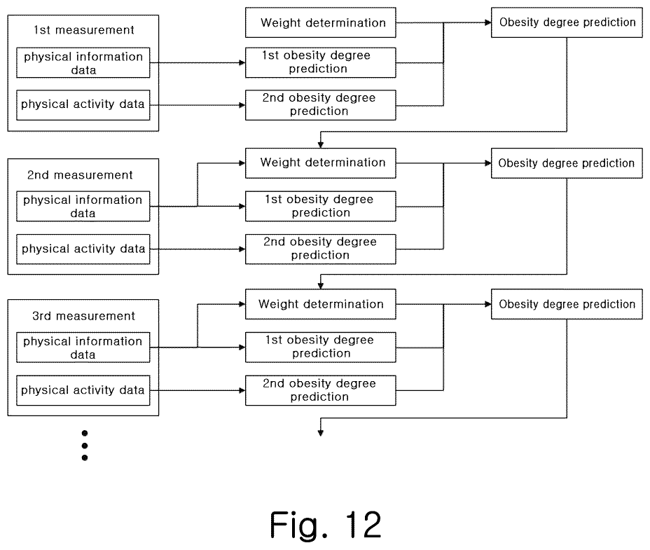

An obesity degree prediction method will be described in more detail with reference to FIG. 12, which is a diagram illustrating an obesity degree prediction process.

A first obesity degree at a predetermined time point (e.g., n months later) is predicted using body information data such as a height and weight, and a second obesity degree at the predetermined time point is predicted using physical activity data such as endurance, agility, balance, and quickness. A final obesity degree is predicted by assigning a weight to each of the first obesity degree and the second obesity degree. The weight in the first measurement may be given as 0.5 as a default value. The second measurement is taken at the predetermined time point, for example, after n months from the first measurement. The body information data and the physical activity data are also obtained in the second measurement, and the first and second obesity degrees after m months, for example, are predicted based on the body information data and physical activity data, respectively. The weight in the second measurement is determined by comparing the predicted obesity degree in the first measurement with the actual obesity degree derived from the height and weight actually measured in the second measurement. The resetting of weights can be understood as training the growth prediction AI model for more accurate predictions. Subsequently, the measurement and prediction may be repeated in the same way. It is described that the degree of obesity is predicted in the present embodiment, but it is also possible to predict height and weight, and then derive the degree of obesity from the height and weight.

Each time whenever the measurement and prediction are repeated, the obesity degree increase rate and the obesity risk are analyzed (S50) and provided to the user (S60).

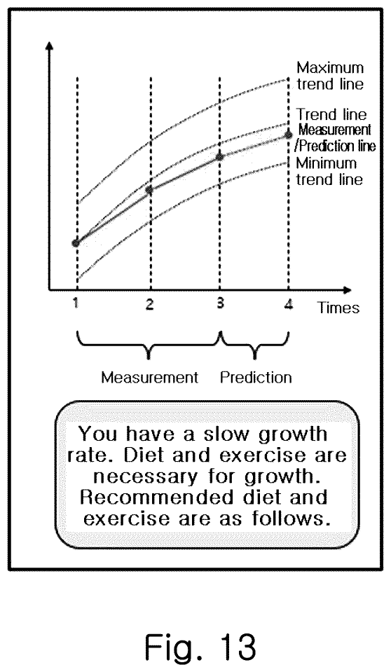

The information provided to the user includes not only the obesity degree increase rate and obesity risk, but also each measured/predicted value of height and weight of the infant, and recommended information such as physical activity and diet. FIG. 13 is a diagram illustrating an example of providing analysis data of information on the height of an infant to a user. In the present embodiment, measurement values at the first to third times are illustrated, and a prediction value at the fourth time is illustrated.

In another embodiment, by using the predicted body information data of an infant, an image model of the infant may be created and the image model may be visually provided to the user.

The system according to the above embodiment may include a terminal for providing physical information data including the gender, height, and weight of the user and physical activity data of the user, and an obesity degree prediction server that is connected to the terminal through a network, analyzes the physical information data and the physical activity data provided from the terminal to predict degree of obesity at a predetermined time point, and provides information on the degree of obesity to the terminal. The obesity degree prediction server may include a storage that stores the data provided from the terminal in a database, an obesity prediction operator that learns the data of the storage through artificial intelligence (AI) to predict obesity at a predetermined time point, and an obesity degree information providing unit that provides the information on the degree of obesity predicted by the obesity degree prediction operator to the terminal. Further, the obesity degree prediction operator may include a first prediction unit that predicts body information at the predetermined time point based on the body information data from the terminal, a second prediction unit that predicts body information at the predetermined time point based on the physical activity data from the terminal, and a weight determination unit that determines a weight applied to each of the predicted values of the first prediction unit and the second prediction unit, and the degree of obesity at the predetermined time point may be predicted based on the predicted values from the first prediction unit and the second prediction unit and the weight from the weight determination unit.

In a method of predicting a degree of obesity of an infant according to still another embodiment of the present disclosure, previously collected body data of infants may be classified into groups, and an obesity degree model for each group may be constructed. The degree of obesity of an infant may be predicted by measuring the body data of the infant, determining which group the infant belongs to, and using the obesity degree model for the corresponding group. In one embodiment, such an obesity model may be generated in advance and stored in a server, and may be downloaded from the server at the request of the client.

In the embodiments of the present disclosure, each component may be implemented by various means, for example, hardware, firmware, software, or a combination thereof. In the case of implementation by hardware, one embodiment of the present disclosure may be implemented by one or more of application specific integrated circuits (ASICs), digital signal processors (DSPs), digital signal processing devices (DSPDs), programmable logic devices (PLDs), field programmed gate array (FPGAs), processors, controllers, microcontrollers, microprocessors, and the like.

In addition, in the case of implementation by firmware or software, one embodiment of the present disclosure may be implemented in the form of a module, procedure, function, and the like that performs the functions or operations described above and may be recorded on a recording medium that is readable through various computer means. Here, the recording medium may include program commands, data files, data structures, and the like alone or in combination. The program commands recorded on the recording medium may be those specially designed and configured for the present disclosure, or those known and available to those skilled in computer software. For example, the recording medium includes a magnetic medium such as a hard disk, a floppy disk and a magnetic tape, an optical medium such as a compact disk read only memory (CD-ROM) and a digital video disk (DVD), a magneto-optical medium such as a floptical disk, and a hardware device specially configured to store and execute program commands such as ROM, RAM, flash memory, and the like. Examples of the program commands may include high-level language codes that can be executed by a computer using an interpreter or the like as well as machine language codes created by a compiler. The hardware devices may be configured to operate as one or more pieces of software to perform the operations of the present disclosure, and vice versa.

Although the specific preferred embodiments of the present disclosure have been described above, the present disclosure is not limited to the specific embodiments described above. Various modifications of the present disclosure may be made by an ordinary skilled person in the art to which the present disclosure pertains without departing from the gist of the present disclosure claimed in the claims, and such modifications are also within the scope of the claims.

The present disclosure as described above can be applied as an AI screening model for large-scale infant health care projects such as infant physical examination and health checkups, and can be used as an obesity risk clinical decision system and a patient management system for hospitals such as children's hospitals, and growth and development clinics.

What is claimed is:

1. A method comprising:
acquiring first data corresponding to body information data including gender, height, and weight of infants or children, and storing the first data in a database;
acquiring second data corresponding to physical activity data for the infants or the children, and storing the second data in the database;
normalizing and categorizing the first and second data;
predicting a degree of obesity after n months through a growth curve of the growth prediction AI model based on the normalized first and second data;
reacquiring the first and second data after the n months and storing the reacquired first and second date in the database or a second database;
training the growth prediction AI model based on the predicted degree of obesity and the reacquired first data;
predicting a degree of obesity after m months through the trained growth prediction AI model; and
analyzing a risk metric of obesity based on the predicted degree of obesity.

2. The method of claim 1, wherein the predicted degree of obesity is a value obtained by computing a predicted value based on the first data and a predicted value based on the second data, and adding the predicted values according to respective weights, and
the training of the growth prediction AI model includes comparing the predicted degree of obesity with the reacquired first data, and adjusting the weights used for predicting the degree of obesity according to the comparison result.

3. The method of claim 2, wherein the weights are ratios of the respective predicted value based on the first data and the second data to the predicted degree of obesity.

4. The method of claim 1, wherein the second data includes data on endurance, agility, balance, and quickness.

5. The method of claim 1, further comprising:
providing the categorized information and/or the analyzed obesity risk information visualized on a display to a user.

6. The method of claim 5, further comprising:
recommending a diet for the infants or the children based on the categorized information or the analyzed obesity risk information.

7. The method of claim 5, further comprising:
recommending physical activity for the infants or the children based on the categorized information or the analyzed obesity risk information.

8. The method of claim 1, wherein the first and second data are acquired annually, quarterly, monthly, or weekly.

9. A system comprising:
a terminal for providing body information data including gender, height, and weight of a user and physical activity data of a user; and
an obesity degree prediction server, connected to the terminal through a network, for analyzing the body information data and the physical activity data provided from the terminal to predict a degree of obesity at a predetermined time point, and providing information on the degree of obesity to the terminal,
wherein the obesity degree prediction server includes:
a storage that stores the data provided from the terminal in a database;
an obesity degree prediction operator that is configured to learn the data in the storage through artificial intelligence (AI) to predict the degree of obesity of the user at the predetermined time point; and
an obesity degree information providing unit that is configured to provide the information on the degree of obesity predicted by the obesity degree prediction operator to the terminal.

10. The system of claim 9, wherein the obesity degree prediction operator includes:
a first prediction unit that predicts body information at the predetermined time point based on the body information data from the terminal;
a second prediction unit that predicts body information at the predetermined time point based on the body activity data from the terminal; and
a weight determination unit that determines a weight assigned to each of predicted values of the first prediction unit and the second prediction unit, and
wherein the degree of obesity at the predetermined time point is predicted based on the predicted values from the first prediction unit and the second prediction unit and the weight from the weight determination unit.

11. The system of claim 10, wherein the weight determination unit adjusts the weight by comparing the predicted degree of obesity at the predetermined time point with the degree of obesity from the body information data obtained at the predetermined time point.

12. The system of claim 9, wherein the obesity degree information providing unit further provides recommended information on diet and physical activity based on the predicted obesity degree information.

13. A non-transitory computer-readable storage device storing instructions that, when executed by a processor, cause the processor to:
acquire first data corresponding to body information data including gender, height, and weight of infants or children, and store the first data in a database;
acquire second data corresponding to physical activity data for the infants or the children, and storing the second data in the database;
normalize and categorize the first and second data;
predict a degree of obesity after n months through a growth curve of the growth prediction AI model based on the normalized first and second data;
reacquire the first and second data after the n months and storing the reacquired first and second date in the database or a second database;
train the growth prediction AI model based on the predicted degree of obesity and the reacquired first data;
predict a degree of obesity after m months through the trained growth prediction AI model; and
analyze a risk metric of obesity based on the predicted degree of obesity.

14. A computer system comprising:
a processor; and
a memory accessible to the processor, the memory storing instructions that are executable by the processor to perform operations comprising:
acquiring first data corresponding to body information data including gender, height, and weight of infants or children, and storing the first data in a database;
acquiring second data corresponding to physical activity data for the infants or the children, and storing the second data in the database;
normalizing and categorizing the first and second data;
predicting a degree of obesity after n months through a growth curve of the growth prediction AI model based on the normalized first and second data;
reacquiring the first and second data after the n months and storing the reacquired first and second date in the database or a second database;
training the growth prediction AI model based on the predicted degree of obesity and the reacquired first data;
predicting a degree of obesity after m months through the trained growth prediction AI model; and
analyzing a risk metric of obesity based on the predicted degree of obesity.
